(19) United States
(12) Patent Application Publication
(10) Pub. No.: US 2025/0099745 A1
(43) Pub. Date: Mar. 27, 2025

(54) PERCUTANEOUS BLOOD PUMP WITH OUTFLOW BLOCKING SLEEVE

(71) Applicant: Boston Scientific Scimed, Inc., Maple Grove, MN (US)

(72) Inventor: Qian Liu, Plymouth, MN (US)

(73) Assignee: Boston Scientific Scimed, Inc., Maple Grove, MN (US)

(21) Appl. No.: 18/894,345

(22) Filed: Sep. 24, 2024

Related U.S. Application Data
(60) Provisional application No. 63/540,183, filed on Sep. 25, 2023.

(51) Int. Cl. A61M 60/833; A61M 60/13; A61M 60/226; A61M 60/416; A61M 60/865

(52) U.S. Cl. CPC A61M 60/833; A61M 60/13; A61M 60/226; A61M 60/416; A61M 60/865

(57) ABSTRACT

An outflow blocking sleeve removably surrounding a blood outlet of a percutaneous blood pump positioned at a distal end of a catheter. The outflow blocking sleeve is configured to prevent blood egress from the blood outlet during insertion of the blood pump into a vasculature at a vascular access site. The blood pump is sized to be passed distally through an elastomeric seal of an introducer sheath into a lumen of an elongate shaft of the introducer sheath while the outflow blocking sleeve is prevented from passing through the elastomeric seal during advancement of the blood pump through the elastomeric seal.

### CROSS-REFERENCE TO RELATED APPLICATIONS

The present application claims the benefit of U.S. Provisional Patent Application Ser. No. 63/540,183, filed Sep. 25, 2023, the disclosure of which is incorporated herein by reference.

### TECHNICAL FIELD

The present disclosure pertains to a percutaneous blood pump including an outflow blocking sleeve. More particularly, the present disclosure pertains a percutaneous blood pump including a sleeve for blocking blood flow through the outflow ports of the blood pump during insertion into a vascular access site.

### BACKGROUND

In various procedures for delivering intravascular medical devices, an introducer sheath is inserted into a blood vessel of a patient, for example a femoral artery, and one or more medical devices may be advanced through the sheath and into the patient's vasculature. In various instances, the medical devices include catheters or other devices, such as a blood pump. In some instances, during advancement of the blood pump through the introducer sheath, there is a moment in which the blood inlet of the blood pump is located distal of a hemostasis valve of the introducer sheath and the blood outlet of the blood pump is located proximal of the hemostasis valve exterior of the patient. In this position, blood may leak through the blood pump to an exterior of the patient through the blood outlet of the blood pump. Thus, there is a need to prevent blood from leaking through the blood pump and out through the blood outlet during insertion of the blood pump across a hemostasis valve into a vasculature of a patient.

### BRIEF SUMMARY

This disclosure provides design, material, manufacturing method, and use alternatives for medical devices, including percutaneous blood pumps and associated devices.

A first example is a system including an introducer sheath and a catheter. The introducer sheath includes a hub, an elongate shaft extending distally from the hub, and an elastomeric seal positioned in the hub. The catheter includes an elongate shaft and a percutaneous blood pump positioned at a distal end of the elongate shaft. The percutaneous blood pump includes a blood inlet, a blood outlet, and an impeller assembly positioned therebetween to pump blood from the blood inlet to the blood outlet during use. An outflow blocking sleeve removably surrounds the blood outlet. The blood pump is sized to be passed distally through the elastomeric seal into a lumen of the elongate shaft of the introducer sheath while the outflow blocking sleeve is prevented from passing through the elastomeric seal during advancement of the blood pump through the elastomeric seal.

Alternatively or additionally to any of the examples above, in another example, the outflow blocking sleeve includes a distal end region having an inner diameter and a proximal end region having a reduced inner diameter less than the inner diameter of the distal end region.

Alternatively or additionally to any of the examples above, in another example, the distal end region tightly surrounds the blood outlet to occlude blood flow therefrom and the proximal end region extends over a distal portion of the elongate shaft of the catheter.

Alternatively or additionally to any of the examples above, in another example, the outflow blocking sleeve has a length of 7 cm or less.

Alternatively or additionally to any of the examples above, in another example, the system includes a guidewire extending out of an outflow window of the blood outlet and extending proximally therefrom along an exterior of the blood pump.

Alternatively or additionally to any of the examples above, in another example, the outflow blocking sleeve surrounds the guidewire extending along the exterior of the blood pump.

Alternatively or additionally to any of the examples above, in another example, the outflow blocking sleeve includes a preferential separation line extending along a length of the outflow blocking sleeve.

Alternatively or additionally to any of the examples above, in another example, the outflow blocking sleeve includes a strip of material configured to separate from a tubular wall of the outflow blocking sleeve when pulled.

Alternatively or additionally to any of the examples above, in another example, the outflow blocking sleeve includes a flared distal end configured to abut a proximal face of the elastomeric sleeve.

Alternatively or additionally to any of the examples above, in another example, the outflow blocking sleeve includes a bulbous distal end configured to abut a proximal face of the elastomeric sleeve.

Another example is a system including a catheter and an outflow blocking sleeve. The catheter includes an elongate shaft and a percutaneous blood pump positioned at a distal end of the elongate shaft. The percutaneous blood pump includes a blood inlet, a blood outlet, an impeller positioned therebetween to pump blood from the blood inlet to the blood outlet during use, and a motor housing positioned proximal of the impeller. The motor housing includes a motor to rotationally drive the impeller. The outflow blocking sleeve tightly surrounds the blood outlet and extends proximally therefrom to surround the motor housing. The outflow blocking sleeve is configured to prevent blood egress from the blood outlet during advancement of the blood pump into a vasculature at a vascular access site.

Alternatively or additionally to any of the examples above, in another example, the outflow blocking sleeve includes a distal end region having an inner diameter and a proximal end region having a reduced inner diameter less than the inner diameter of the distal end region.

Alternatively or additionally to any of the examples above, in another example, the distal end region tightly surrounds the blood outlet to occlude blood flow therefrom and the proximal end region extends over a distal portion of the elongate shaft of the catheter proximal of the motor housing.

Alternatively or additionally to any of the examples above, in another example, the outflow blocking sleeve has a length of 7 cm or less.

Alternatively or additionally to any of the examples above, in another example, the system includes a guidewire extending out of an outflow window of the blood outlet and extending proximally therefrom along an exterior of the motor housing, wherein the outflow blocking sleeve surrounds the guidewire extending along the exterior of the motor housing.

Another example is a method of percutaneously inserting a blood pump into a vasculature at a vascular access site. The method includes advancing a catheter, including the blood pump positioned at a distal end of an elongate shaft of the catheter, into an introducer sheath having an elastomeric seal. The blood pump includes a blood inlet, a blood outlet, and an impeller assembly positioned therebetween to pump blood from the blood inlet to the blood outlet during use. The blood pump is advanced through the elastomeric seal while an outflow blocking sleeve occludes the blood outlet when the blood outlet is located proximal of the elastomeric seal and the blood inlet is located distal of the elastomeric seal such that blood egress from the blood outlet is prevented. Thereafter, the blood pump is further advanced through the elastomeric seal while the outflow blocking sleeve abuts a proximal face of the elastomeric sleeve and remains exterior of the elastomeric seal such that the blood outlet passes through the elastomeric seal to a location distal of the elastomeric seal.

Alternatively or additionally to any of the examples above, in another example, the blood pump is advanced over a guidewire extending out of an outflow window of the blood outlet and the guidewire extends proximally therefrom along an exterior of the blood pump.

Alternatively or additionally to any of the examples above, in another example, the outflow blocking sleeve surrounds the guidewire extending along the exterior of the blood pump.

Alternatively or additionally to any of the examples above, in another example, the method includes laterally removing the outflow blocking sleeve from the catheter after the blood outlet of the blood pump is advanced to a location distal of the elastomeric seal.

Alternatively or additionally to any of the examples above, in another example, the outflow blocking sleeve is splittable along its length to separate the outflow blocking sleeve from the catheter.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a perspective view of an exemplary catheter including a percutaneous blood pump;

FIG. 2 shows the distal end region of the catheter of FIG. 1 including the percutaneous blood pump;

FIG. 3 is a side view of a portion of the percutaneous blood pump of FIG. 1;

FIG. 4 is a cross-sectional view of the portion of the percutaneous blood pump of FIG. 3, with a blocking sleeve disposed thereon;

FIG. 5 is a perspective view of an exemplary introducer sheath;

FIGS. 6-8 illustrate steps of inserting the percutaneous blood pump of FIG. 1 through the introducer sheath of FIG. 5 at a vascular access site; and

FIGS. 9-14 illustrate various features of a blocking sleeve for use with the percutaneous blood pump of FIG. 1.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

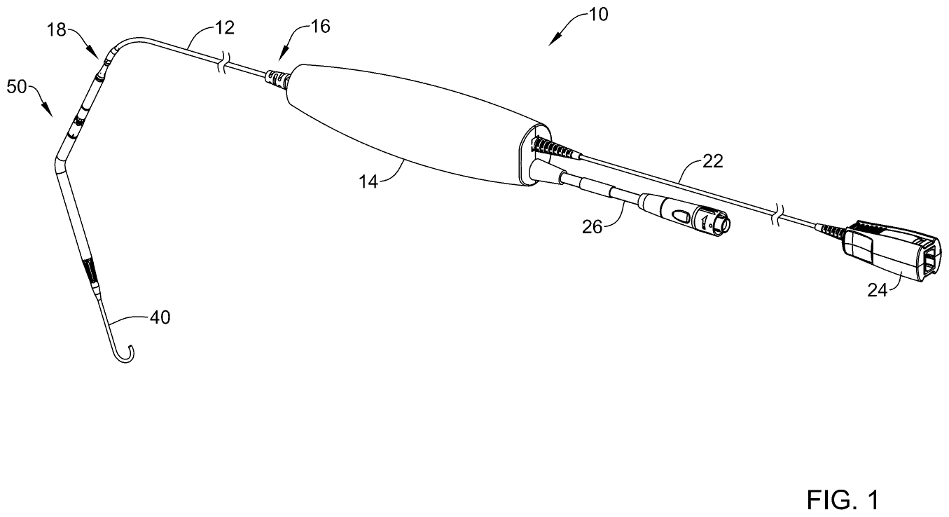

FIG. 1 illustrates a perspective view of a catheter 10 including a percutaneous blood pump 50 located at a distal end region thereof. The catheter 10 may be coupled to or include the blood pump 50, with an elongate shaft 12 of the catheter 10 extending proximally from the percutaneous blood pump 50 and a distal tip 40 extending distally from the blood pump 50. For instance, a proximal end 16 of the elongate shaft 12 may be coupled to a control module 14 and a distal end 18 of the elongate shaft 12 may be coupled to the percutaneous blood pump 50. An electrical cable 22 may extend from the control module 14 to a connector 24 at a proximal end thereof. The connector 24 may be configured to be connected to a controller (not shown) for controlling the blood pump 50, such as providing electrical power to the blood pump 50. The catheter 10 may also include an extension 26 connectable to the controller for sending and/or receiving signals, such as from one or more sensors during operation of the blood pump 50.

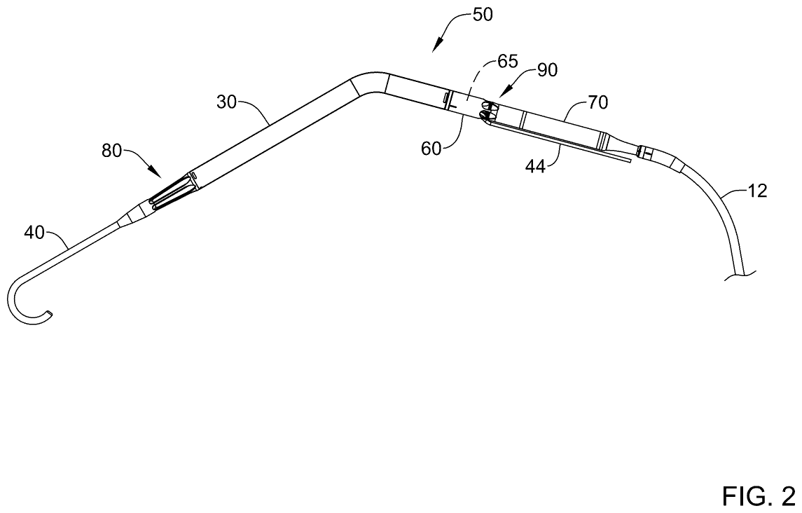

Additional features of the blood pump 50 are illustrated in FIG. 2. The blood pump 50 may generally include a flexible cannula 30, an impeller housing 60, and a motor housing 70. In some embodiments, the flexible cannula 30, the impeller housing 60 and/or the motor housing 70 may be integrally or monolithically constructed. In other instances, the flexible cannula 30, the impeller housing 140 and/or the motor housing 70 may be separate components. The impeller housing 60 carries an impeller assembly 65 therein. The impeller assembly 65 may include an impeller secured to an impeller shaft that rotates relative to the impeller housing 60 to drive blood through the blood pump 50. In some embodiments, the impeller shaft and the impeller of the impeller assembly 65 may be integrally formed, whereas, in other embodiments the impeller shaft and the impeller may be separate components.

Rotation of the impeller causes blood to flow from a blood inlet 80 of the blood pump 50, such as at a distal end of the flexible cannula 30, through the flexible cannula 30 and the impeller housing 60, and out of a blood outlet 90 proximal of the impeller, such as through a sidewall formed on the impeller housing 60. In some instances, the blood inlet 80 may include a plurality of blood inlet windows arranged around a circumference of the blood pump 50 (e.g., the flexible cannula 30). In some instances, the blood outlet 90 may include a plurality of blood outflow windows arranged around a circumference of the impeller housing 60. In other embodiments, the inlet 80 and/or the outlet 90 may be formed on other portions of the blood pump 50.

With continued reference to FIG. 2, the motor housing 70 carries a motor configured to rotatably drive the impeller of the impeller assembly 65 relative to the impeller housing 60. Electrical power may be supplied to the motor through wiring extending through the elongate shaft 12, for example. In some instances, the motor may be physically connected to the impeller. For example, in some embodiments the impeller may be mounted on the drive shaft of the motor. In other embodiments, the impeller shaft may be directly or indirectly coupled to the drive shaft of the motor. In some instances, the drive assembly may include a magnetic coupling between the motor and the impeller. For example, a driving magnet may be mounted on the drive shaft of the motor. Rotation of the driving magnet causes rotation of a driven magnet, which is connected to the impeller assembly 65. More specifically, in embodiments incorporating an impeller shaft, the impeller shaft and the impeller of the impeller assembly 65 are configured to rotate with the driven magnet. In other embodiments, the motor may be coupled to the impeller assembly 65 via other components.

Additionally shown in FIG. 2, the blood pump 50 may include a guidewire loading aid 44 to facilitate loading the catheter 10 onto a guidewire for introduction of the blood pump 50 into a vasculature. For example, the guidewire loading aid 44 may be a tubular member extending through at least a portion of the length of the blood pump 50 through which a guidewire may be routed when loading the catheter onto the guidewire. In some instances, the guidewire loading aid 44 may have a distal end (not shown) located within the blood pump 50 and a proximal end located exterior of the blood pump 50. A proximal end portion of the guidewire loading aid 44 may extend out through one of the outflow windows of the blood outlet 90 and extend proximally therefrom. In some instances, the proximal end portion of the guidewire loading aid 44 may extend along an exterior of the motor housing 70, located proximal of the blood outlet 90. A guidewire, inserted through a guidewire lumen of the distal tip 40, may be guided into the lumen of the guidewire loading aid 44 and advanced proximally along the impeller assembly 65 and out through one of the outflow windows of the blood outlet 90 within the lumen of the guidewire loading aid 44. Once the guidewire has been properly threaded through the blood pump 50, the guidewire loading aid 44 may be removed, leaving the guidewire tracked through the blood pump 50 such that the catheter 10 may be advanced over the guidewire into a vasculature.

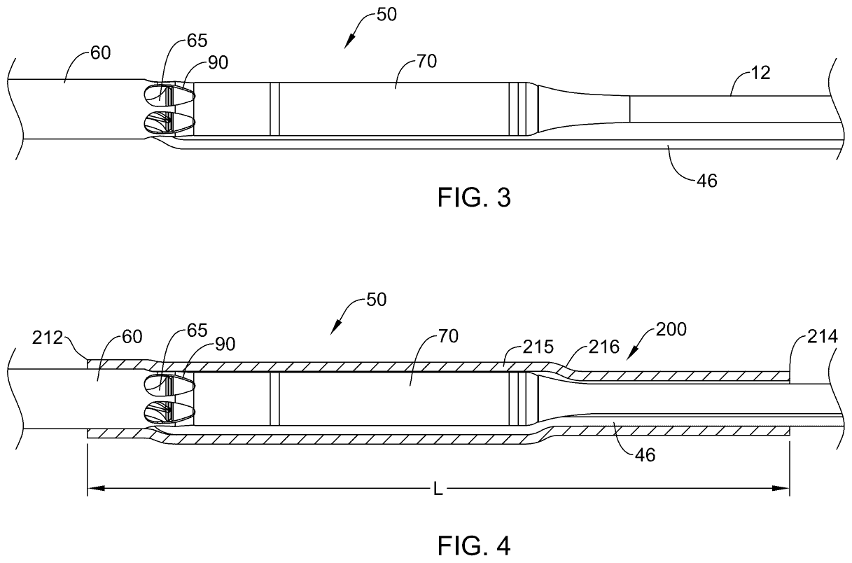

FIG. 3 is a side view of a portion of the catheter 10 illustrating a portion of the percutaneous blood pump 50 and a guidewire 46 extending out of one of the outflow windows of the blood outlet 90 of the impeller housing 60 and extending along an exterior of the motor housing 70 and the elongate shaft 12. A proximal end of the impeller of the impeller assembly 65 is also visible through the outflow windows of the blood outlet 90.

To prevent blood from leaking through the cannula of the blood pump 50 and out through the outflow windows of the blood outlet 90 during insertion of the blood pump 50 into a vasculature of a patient, it may be desirable to block or obstruct the blood outlet 90 to prevent egress of blood therefrom. As such, an outflow blocking sleeve 200, shown in FIG. 4, may be positioned across the blood outlet 90 to block or obstruct the outflow windows of the blood outlet 90. The outflow blocking sleeve 200 may be a tubular member 215 having a distal end 212 and a proximal end 214. The outflow blocking sleeve 200 may be formed of a polymer material, such as an elastomeric polymer. Some suitable materials include fluorinated ethylene propylene (FEP), high-density polyethylene (HDPE), polyether-block amide (e.g., PEBAX®), polyurethane, polyamide (e.g., VESTAMID®), etc. The tubular member 215 may have an inner surface tightly conforming to a portion of the blood pump 50 that the outflow blocking sleeve 200 surrounds. For example, the distal end of the tubular member 215 of the outflow blocking sleeve 200 may extend distal of the blood outlet 90 such that a distal end region of the tubular member 215 tightly surrounds the impeller housing 60 defining the blood outlet 90 in order to occlude or block the outflow windows of the blood outlet 90. The outflow blocking sleeve 200 may be formed of a polymer material having a desired elasticity to stretch or conform to the outer surface of the impeller housing 60 and/or the motor housing 70. As shown in FIG. 4, the outflow blocking sleeve 200 may also surround the guidewire 46 extending out from the outflow window of the blood outlet 90 along an exterior of the motor housing 70. In some instances, the inner surface of the outflow blocking sleeve 200 may exert a force against the guidewire 46 to press the guidewire 46 against the exterior surface of the motor housing 70, while allowing the blood pump 50 to move axially along the guidewire 46. In some instances, the tubular member 215 may have a tapered or necked down region 216 located at an intermediate location along the tubular member 215 between a distal end region extending to the distal end 212 and a reduced diameter proximal end region extending to the proximal end 214. The tapered region 216 may be positioned at a proximal end of the motor housing 70, where the elongate shaft 12 is coupled to the motor housing 70 at a connection, such that the reduced diameter proximal end region of the tubular member 215 of the outflow blocking sleeve 200 closely conforms to an exterior surface of the elongate shaft 12 proximal of the connection with the motor housing 70.

The outflow blocking sleeve 200 may have a length L measured from the distal end 212 to the proximal end 214. In some embodiments, the length L may be in the range of 0.5 cm to 10 cm, in the range of 0.5 cm to 8 cm, in the range of 0.5 cm to 6 cm, in the range of 1.0 cm to 6 cm, or in the range of 1.0 cm to 5 cm, for example. The length L may be 8 cm or less, 7 cm or less, 6 cm or less, or 5 cm or less, in some instances. In some instances, the outflow blocking sleeve 200 may have a length L greater than the length of the motor housing 70. In some instances, the motor housing 70 may have a length of about 4 cm, and thus the outflow blocking sleeve 200 may have a length of greater than 4 cm, such that the distal end of the outflow blocking sleeve 200 extends distal of the motor housing 70 to block or occlude the blood outlet 90 and the proximal end of the outflow blocking sleeve 200 extends proximal of the motor housing 70 to surround the elongate shaft 12.

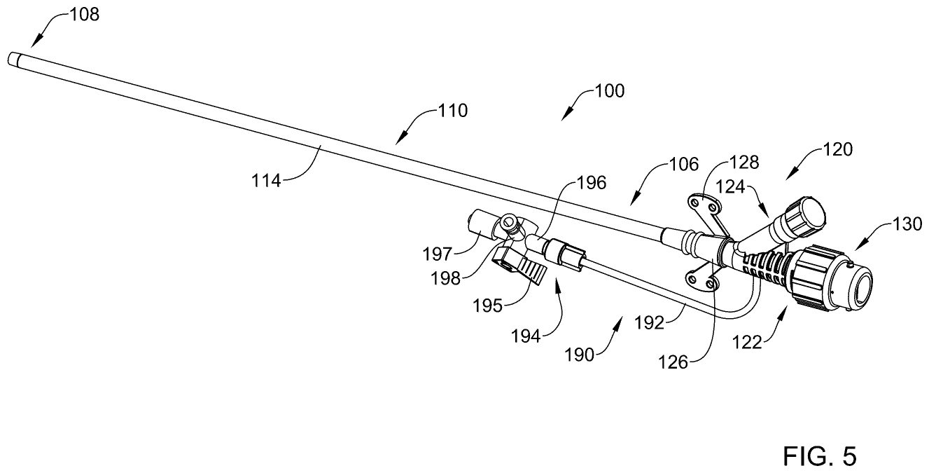

The outflow blocking sleeve 200 may be slidably positioned over the elongate shaft 12 of the catheter 10 such that the outflow blocking sleeve 200 may be selectively slid over the blood outlet 90 to block blood flow therethrough and selectively removed from the blood outlet 90 to permit blood flow therethrough. For example, it may be desirable to position the outflow blocking sleeve 200 over the outflow windows of the blood outlet 90 during introduction of the blood pump 50 into the vasculature through a vascular access site to prevent blood from the patient from flowing through the blood pump 50 and out the blood outlet 90. For example, during the introduction process, at the moment in which the blood inlet 80 is exposed to blood in the vasculature of the patient and the blood outlet 90 is still exterior of the vasculature of the patient, a pathway through the blood pump 50 from the blood inlet 80 to the blood outlet 90 may allow for blood flow therethrough. If the blood outlet 90 is not obstructed during this process, the blood may exit the blood outlet 90 exterior of the patient. Accordingly, the outflow blocking sleeve 200 may be used to occlude the blood outlet 90 as the blood pump 50 is inserted into the vasculature of the patient through a vascular access site. In some instances, the blood pump 50 may be inserted through an introducer sheath at the vascular access site. One such introducer sheath is shown in FIG. 5.

FIG. 5 is a perspective view of an introducer sheath 100 for use in providing vascular access for introducing the percutaneous blood pump of FIG. 1 into a vasculature at a vascular access site. The introducer sheath 100 may include a hub 120, as well as an elongate shaft 114 extending distally from the hub 120 to a distal end region 108 and defining the body portion 110 of the sheath 100. The sheath 100 may also include a flush line 190 extending from the hub 120. The flush line 190 may include a tubular member 192 extending from the hub 120 and in fluid communication with a lumen of the hub 120. The tubular member 192 may extend to a stopcock 194, such as a three-way stopcock. The stopcock 194 may include a first leg 196 coupled to the tubular member 192, a second leg 197, a third leg 198, and a lever 195 that is rotatable to selectively open/close fluid access between the first, second and/or third legs 196, 197, 198. Each of the legs 196, 197, 198 may include a connector, such as a luer connector, as desired.

The hub 120 may also include a strain relief 126 configured to provide a transition in flexibility along the proximal end region 106 of the elongate shaft 114. The strain relief 126 may include a body attached to a main body of the hub 120, as will be described further herein. The strain relief 126 may include one or more suture pads 128 extending outward therefrom. For example, the strain relief 126 may include first and second suture pads 128 extending from opposite sides of the strain relief 126. The suture pads 128 may facilitate securing the hub 120 against the patient once the introducer sheath 100 has been positioned in the blood vessel of the patient. For example, each suture pad 128 may include at least one opening extending therethrough for receiving a suture used to suture the hub 120 to the skin of the patient.

The hub 120 may include a main port 122 and a side port 124 extending from the main port 122. In some instances, the side port 124 may extend at an acute angle from the main port 122. The main port 122 and/or the side port 124 may provide access to one or more lumens extending through the body portion 110 (e.g., through the elongate shaft 114) of the sheath 100. In some instances, the main port 122 may be a tightening port 130.

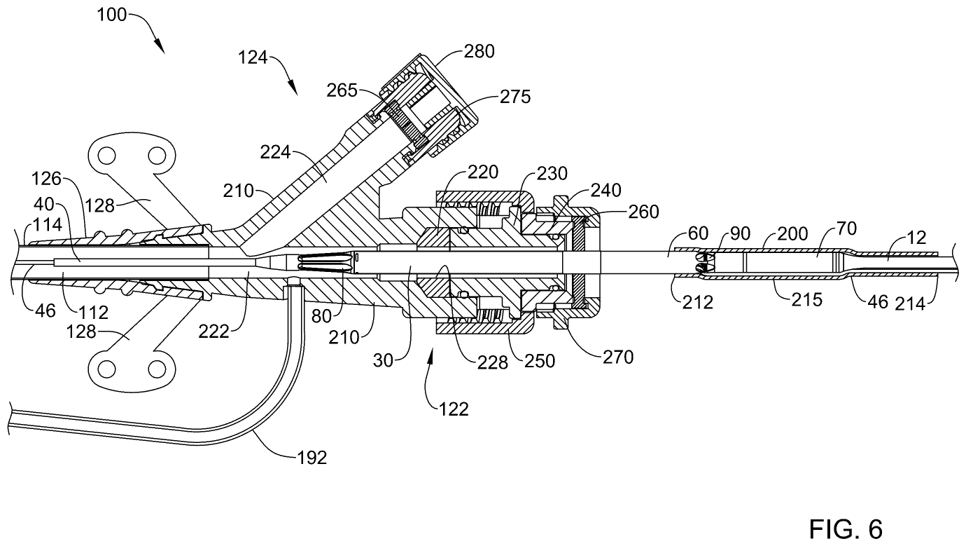

Further details of the components of the hub 120 are shown in the cross-sectional views of FIGS. 6-8. The main port 122 of hub 120 may include a hub body 210. In some instances, the hub body 210 may be a molded, one-piece structure. For example, the hub body 210 may be molded from a polymeric material. In other instances, the hub body 210 may be formed of two or more components attached together. In some instances, the hub body 210 may also include or be connected to the hub body of the side port 124.

The side port 124 may include a passage 224 extending therethrough and an elastomeric seal 265 positioned along the passage 224 of the side port 124. For example, hub body 210 may define the passage 224. The elastomeric seal 265 may be a slit valve (e.g., a cross-slit valve), a dome valve, a duckbill valve, or any other desired valve configured to seal around an elongate shaft of a medical device when passed therethrough. In some instances, the elastomeric seal 265 may include one or more slits (e.g., crossing slits) extending entirely through the seal wall and/or one or more slits (e.g., crossing slits) extending only partially through the seal wall. For instance, the elastomeric seal 265 may be a cross-slit valve having a first slit extending into the wall of the seal from a first side of the seal but not extend entirely through the wall of the seal and a second slit extending into the wall of the seal from a second, opposite side of the seal but not extend entirely through the wall of the seal. The first slit may intersect the second slit within the wall of the valve. In some instances, the first slit may be arranged perpendicular to the second slit. The elastomeric seal 265 may be formed of any desired flexible material, such as silicone, polyurethane, etc.

The side port 124 may also include a side port lid 275 connectable to the hub body 210. The elastomeric seal 265 may be housed in the side port lid 275 such that the elastomeric seal 265 is captured between an upper surface of the hub body 210 and an internal surface of the side port lid 275.

The side port 124 may also include a side port cap 280 configured to threadably engage the side port lid 275. For example, the side port cap 280 may include internal threads configured to threadably engage external threads in the side port lid 275. Removal of the side port cap 280 may allow access to the elastomeric seal 265 such that a medical device may be passed through the elastomeric seal 265 into the side port passageway 224.

The main port 122, may include a passage 222 extending therethrough. For example, the hub body 210 may define the passage 222. The passage 224 of the side port 124 may converge with the passage 222 of the main port 122 within the hub body 210. The passage 222 and/or the passage 224 may be in fluid communication with the lumen 112 of the sheath 100 extending to a distal opening of the elongate shaft 114.

The main port 122 may include a primary seal and a secondary seal spaced apart from the primary seal along a length of the main port 122. For example, the main port 122 may include a compressible seal (e.g., Tuohy seal) 220 and an elastomeric seal 260 spaced apart from the compressible seal 220. The main port 122, which may be considered a tightening port, may further include a pusher 230, a holder 240, a lock nut 250, and/or a main port lid 270. Rotation of the lock nut 250 may actuate the pusher 230 toward/away from the compressible seal 220 to adjust the size of the opening 228 through the compressible seal 220. As described further herein, the compressible seal 220 may be movable between an open state, allowing a medical device to pass through the opening 228, and a closed state, sealing the compressible seal 220 around the medical device. The compressible seal 220 may be formed of any desired flexible material, such as silicone, polyurethane, etc.

The holder 240 may be secured to the hub body 210 with a rim of the holder 240 juxtaposed with the proximal end face of the hub body 210. For instance, the main port lid 270 may be connectable to the hub body 210 with the holder 240 therebetween, thereby securing the holder 240 relative to the hub body 210.

The elastomeric seal 260 may be housed in the main port lid 270 such that the elastomeric seal 260 is captured between an end surface of the holder 240 and an internal surface of the main port lid 270. The main port lid 270 may extend distally beyond the elastomeric seal 260 such that a distal end region of the main port lid 270 surrounds the holder 240. The elastomeric seal 260 may be a slit valve (e.g., a cross-slit valve), a dome valve, a duckbill valve, or any other desired valve configured to seal around an elongate shaft of a medical device when passed therethrough. In some instances, the elastomeric seal 260 may include one or more slits (e.g., crossing slits) extending entirely through the seal wall and/or one or more slits (e.g., crossing slits) extending only partially through the seal wall. For instance, the elastomeric seal 260 may be a cross-slit valve having a first slit extending into the wall of the seal from a first side of the seal but not extend entirely through the wall of the seal and a second slit extending into the wall of the seal from a second, opposite side of the seal but not extend entirely through the wall of the seal. The first slit may intersect the second slit within the wall of the valve. In some instances, the first slit may be arranged perpendicular to the second slit. The elastomeric seal 260 may be formed of any desired flexible material, such as silicone, polyurethane, etc. The pusher 230 may be positioned entirely between the compressible seal 220 and the elastomeric seal 260.

The lock nut 250 may be assembled such that the lock nut 250 surrounds a proximal portion of the hub body 210. The lock nut 250 may include internal threading threadably engaged with external threading provided on an exterior of the proximal portion of the hub body 210.

As shown in the cross-sectional view of FIGS. 6-8, the components of the main port 122 may form a hemostasis valve. Accordingly, rotating the lock nut 250 in a first rotational direction causes the lock nut 250 to travel distally relative to the hub body 210, and rotating the lock nut 250 in a second, opposite rotational direction causes the lock nut 250 to travel proximally relative to the hub body 210. The lock nut 250 may be configured to engage the pusher 230 to exert axial movement thereon when the lock nut 250 is rotated. For example, as the lock nut 250 is rotated, and thus travels axially along the hub body 210, the lock nut drives the pusher 230 in an axial direction. For example, as the lock nut 250 is rotated in a first rotational direction relative to the hub body 210, the lock nut 250 travels distally, urging the pusher 230 to travel distally relative to the hub body 210 to exert a compressive force on the compressible seal 220 to thereby reduce the diameter of the opening 228 through the compressible seal 220. As the lock nut 250 is rotated in a second rotational direction relative to the hub body 210, the lock nut 250 travels proximally, permitting the pusher 230 to travel proximally relative to the hub body 210 to reduce and/or remove the compressive force on the compressible seal 220 to thereby increase the diameter of the opening 228 through the compressible seal 220.

In other embodiments, the position of the elastomeric seal 260 and the compressible seal 220 may be revised, with the elastomeric seal 260 located distal of the compressible seal 220. In some such embodiments, the pusher 230 may be positioned entirely between the compressible seal 220 and the elastomeric seal 260. In such an instance, as the lock nut 250 is rotated in a first rotational direction relative to the hub body 210, the lock nut 250 may travel proximally, urging the pusher 230 to travel proximally relative to the hub body 210 to exert a compressive force on the compressible seal 220 (located proximal of the pusher 230) to thereby reduce the diameter of the opening 228 through the compressible seal 220. As the lock nut 250 is rotated in a second rotational direction relative to the hub body 210, the lock nut 250 travels distally, permitting the pusher 230 to travel distally relative to the hub body 210 to reduce and/or remove the compressive force on the compressible seal 220 to thereby increase the diameter of the opening 228 through the compressible seal 220. Other arrangements are contemplated for positioning the pusher 230 relative to the compressible seal 220 to apply a compressive force thereto.

FIGS. 6-8 further illustrate a process of inserting the blood pump 50 of the catheter 10 into a vasculature of a patient through the introducer sheath 100. FIGS. 6-8 show the components of the blood pump 50 in plan view, while components of the introducer sheath 100 and the outflow blocking sleeve 200 are shown in cross-section.

In use, the blood pump 50 of the catheter 10 may be inserted through the introducer sheath 100 along a guidewire 46. For example, the blood pump 50 may be advanced through the elastomeric seal 260, through the lumen of the pusher 230, through the opening 228 of the compressible seal 220 and into the passageway 222 of the main port 122, and into the lumen of the elongate shaft 114 of the introducer sheath 100. For example, as shown in FIG. 6, the distal tip 40 and the blood pump 50 may be passed through an opening of the elastomeric seal 260 and cause the elastomeric seal 260 to flex and/or deform to permit the blood pump 50 to be advanced therethrough. The elastomeric seal 260 may seal around an outer perimeter of the cannula 30 of the blood pump 50 and substantially prevent blood from escaping from the main port 122.

As shown in FIG. 6, during advancement of the blood pump 50 through the introducer sheath 100, there is a moment in which the blood inlet 80 is located distal of the compressible seal 220 and/or the elastomeric seal 260, while the blood outlet 90 is located proximal of the compressible seal 220 and/or the elastomeric seal 260, exterior of the patient. While in this position, the outflow blocking sleeve 200 blocks or occludes the outflow windows of the blood outlet 90 from blood egress therefrom, which would otherwise flow out through the blood outlet 90.

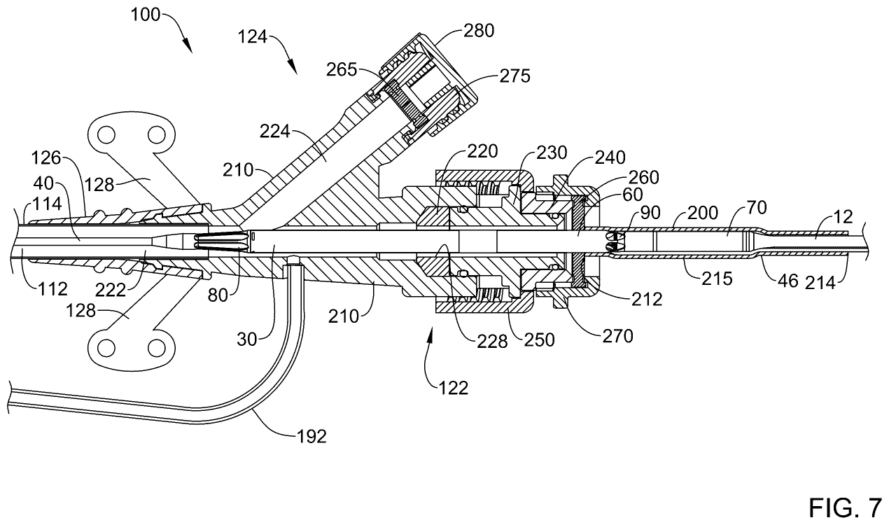

The blood pump 50, along with the outflow blocking sleeve 200, may be advanced distally together until the distal end 212 of the outflow blocking sleeve 200 abuts the proximal face of the elastomeric seal 260, shown in FIG. 7. Since the outflow windows of the blood outlet 90 are still proximal of the elastomeric seal 260, the presence of the outflow blocking sleeve 200 extending distal of (e.g., across) the outflow windows of the blood outlet 90 and occluding the outflow windows of the blood outlet 90 prevents blood egress therefrom, maintaining hemostasis.

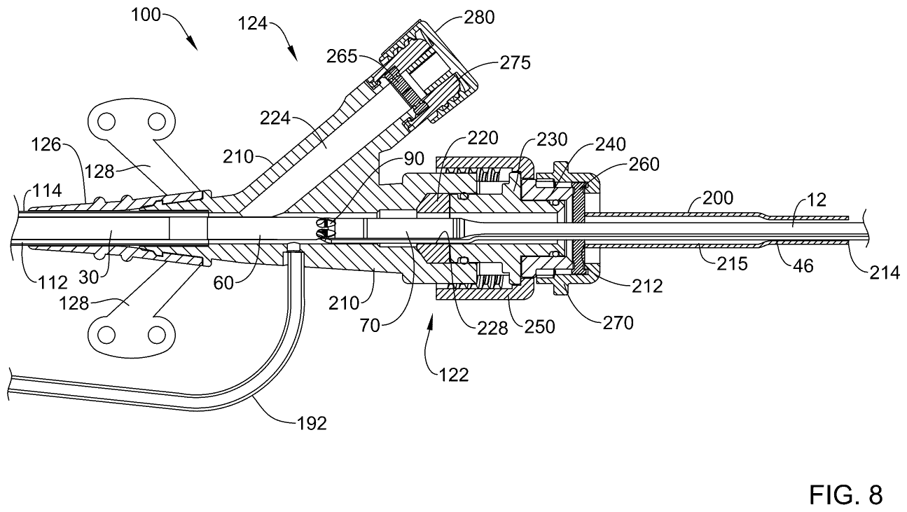

Upon further distal advancement of the blood pump 50 relative to the introducer sheath 100 into the vasculature of the patient, the outflow blocking sleeve 200 may remain stationary relative to the introducer sheath 100. In other words, the blood pump 50 may be further advanced distally through the elastomeric seal 260 while the entire length of the outflow blocking sleeve 200 remains proximal of the elastomeric seal 260, as shown in FIG. 8. Accordingly, the outflow blocking sleeve 200 does not pass through the elastomeric seal 260 as the blood pump 50 is advanced through the elastomeric seal 260 of the introducer sheath 100. Thus, the outflow blocking sleeve 200 may occlude the outflow windows of the blood outlet 90 while the blood outlet 90 is proximal of the elastomeric seal 260, and thereafter, the presence of the elastomeric seal 260 sealingly surrounding the outer perimeter of the motor housing 70 of the blood pump 50 proximal of the blood outlet 90 substantially prevents blood from escaping from the main port 122, maintain hemostasis.

The elongate shaft 12 of the catheter 10 may be further advanced through the introducer sheath 100 into the body of the patient to position the blood pump 50 in a desired location, such as across the aortic valve between the left ventricle of the heart and the aorta. Once the blood pump 50 is positioned at a desired location within the vasculature of the patient (e.g., within the heart of the patient), the compressible seal 220 may be compressed or tightened around the perimeter of the elongate shaft 12 of the catheter 10 to prevent blood from leaking past the elongate shaft 12 and out of the main port 122 and/or lock the elongate shaft 12 of the catheter 10 from axial movement relative to the hub 120. For instance, as discussed above, the lock nut 250 may be rotated to distally advance the pusher 230, which in turn exerts a compressive force against the compressible seal 220, reducing the diameter of the opening 228 through the compressible seal 220, to seal the compressible seal 220 around the elongate shaft 12 of the catheter 10, maintain hemostasis.

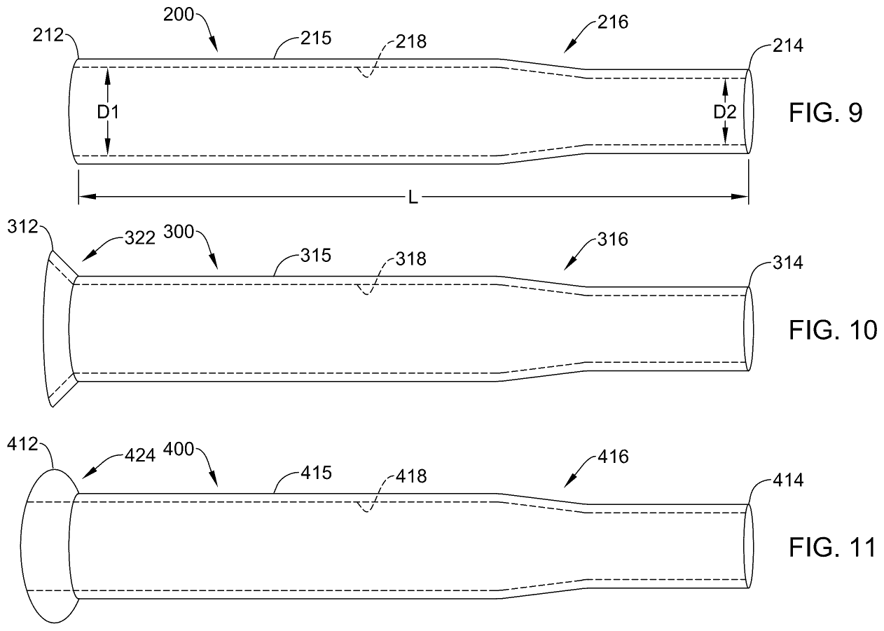

FIGS. 9-14 illustrate various features of the outflow blocking sleeve 200 and/or variations of the outflow blocking sleeve 200. It is noted that the outflow blocking sleeve may include any combination of the various features and/or variations described herein.

As shown in FIG. 9, the outflow blocking sleeve 200 may be a tubular member 215 having a distal end 212, a proximal end 214, and an inner wall surface 218 defining a lumen extending therethrough. The outflow blocking sleeve 200 may have a length L measured from the proximal end 214 to the distal end 212. Exemplary lengths L of the outflow blocking sleeve 200 have been noted above. In some instances, the outflow blocking sleeve 200 may include a tapered region 216 located between a distal end region extending to the distal end 212 and a reduced diameter proximal end region extending to the proximal end 214. Thus, the distal end region may have an inner diameter D1 and the proximal end region may have an inner diameter D2, smaller than the inner diameter D1. In some instances, the tubular member 215 defining the outflow blocking sleeve 200 may have a wall thickness of 0.005 inches to 0.015 inches, or about 0.010 inches, for example. The tubular member 215 may be flexible or compliant, allowing the tubular member 215 to radially stretch to fit around the rigid housing of the blood pump 50 defining the blood outlet 90.

Another illustrative embodiment of the outflow blocking sleeve 200, depicted as outflow blocking sleeve 300, is shown in FIG. 10. The outflow blocking sleeve 300 may be similar to the outflow blocking sleeve 200 in many aspects. For example, the outflow blocking sleeve 300 may be a tubular member 315 having a distal end 312, a proximal end 314, and an inner wall surface 318 defining a lumen extending therethrough. The outflow blocking sleeve 300 may include a tapered region 316 located between a distal end region extending to the distal end 312 and a reduced diameter proximal end region extending to the proximal end 314. The outflow blocking sleeve 300 may include a flared distal region 322 flaring radially outward to a larger outer diameter at the distal end 312. As shown in FIG. 10, the inner diameter of the tubular member 315, defining the lumen, may also taper radially outward in a distal direction through the flared distal region 322. The flared distal region 322 may be configured to abut the proximal face of the elastomeric seal 260 to further inhibit the outflow blocking sleeve 300 from passing distally through the elastomeric seal 260.

Another illustrative embodiment of the outflow blocking sleeve 200, depicted as outflow blocking sleeve 400, is shown in FIG. 11. The outflow blocking sleeve 400 may be similar to the outflow blocking sleeve 200 in many aspects. For example, the outflow blocking sleeve 400 may be a tubular member 415 having a distal end 412, a proximal end 414, and an inner wall surface 418 defining a lumen extending therethrough. The outflow blocking sleeve 400 may include a tapered region 416 located between a distal end region extending to the distal end 412 and a reduced diameter proximal end region extending to the proximal end 414. The outflow blocking sleeve 400 may include a bulbous distal region 424 having a larger outer diameter at the distal end 412 compared to the remainder of the length of the tubular member 415. As shown in FIG. 11, the inner diameter of the tubular member 415, defining the lumen, may be constant throughout the distal end region, including through the bulbous distal region 424, while the proximal end region may include a reduced inner diameter. The bulbous distal region 424 may be configured to abut the proximal face of the elastomeric seal 260 to further inhibit the outflow blocking sleeve 400 from passing distally through the elastomeric seal 260.

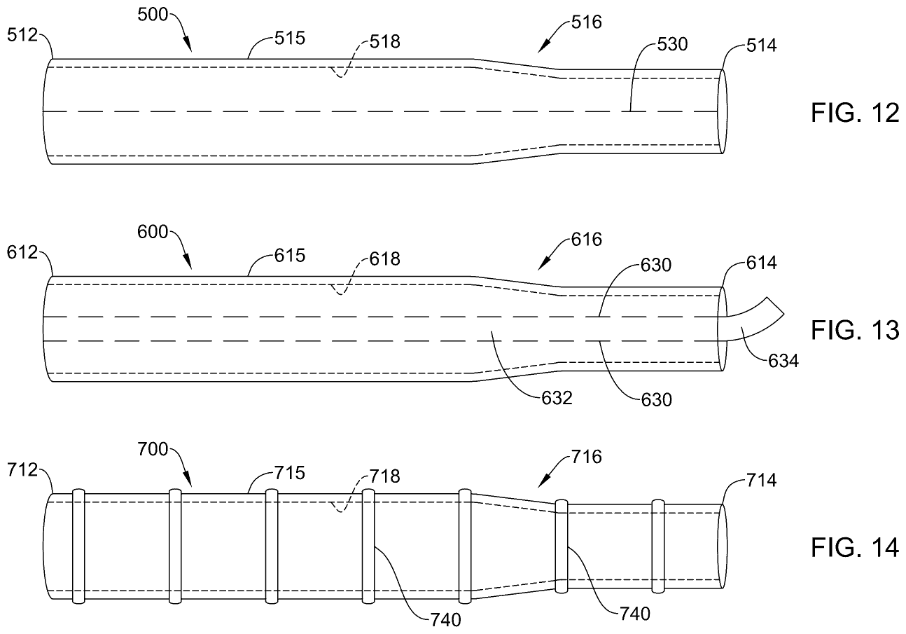

Another illustrative embodiment of the outflow blocking sleeve 200, depicted as outflow blocking sleeve 500, is shown in FIG. 12. The outflow blocking sleeve 500 may be similar to the outflow blocking sleeve 200 in many aspects. For example, the outflow blocking sleeve 500 may be a tubular member 515 having a distal end 512, a proximal end 514, and an inner wall surface 518 defining a lumen extending therethrough. The outflow blocking sleeve 500 may include a tapered region 516 located between a distal end region extending to the distal end 512 and a reduced diameter proximal end region extending to the proximal end 514. The outflow blocking sleeve 500 may include a preferential separation line 530. In some instances, the outflow blocking sleeve 500 may include a plurality of preferential separation lines 530. The preferential separation line(s) 530 may be arranged at desired intervals along the length and/or circumference of the tubular member 515. The tubular member 515 of the outflow blocking sleeve 500 may be configured to preferentially separate along the preferential separation line 530 for removal from the blood pump 50 and/or elongate shaft 12 of the catheter 10. For instance, the preferential separation line 530 may include one or more perforations, apertures, notches, slits, slots, channels, grooves, voids, stress risers, weakened areas, permitting one region of the tubular member 515 to separate from another region of the tubular member 515 along the length of the tubular member 515 such that a longitudinal edge of the tubular member 515 faces another longitudinal edge of the tubular member 515 along the length of the outflow blocking sleeve 500. Once the outflow blocking sleeve 500 has been split along preferential separation line 530, the outflow blocking sleeve 500 may be removed laterally from the blood pump 50 and/or elongate shaft 12 of the catheter 10 by passing the blood pump 50 and/or elongate shaft 12 of the catheter 10 through the gap formed between the adjacent longitudinal edges of the tubular member 515.

Another illustrative embodiment of the outflow blocking sleeve 200, depicted as outflow blocking sleeve 600, is shown in FIG. 13. The outflow blocking sleeve 600 may be similar to the outflow blocking sleeve 200 in many aspects. For example, the outflow blocking sleeve 600 may be a tubular member 615 having a distal end 612, a proximal end 614, and an inner wall surface 618 defining a lumen extending therethrough. The outflow blocking sleeve 600 may include a tapered region 616 located between a distal end region extending to the distal end 612 and a reduced diameter proximal end region extending to the proximal end 614. The outflow blocking sleeve 600 may include a tear strip 632 extending along the tubular member 615. The tear strip 632 may extend longitudinally and/or helically along the tubular member 615 from the proximal end 614 to the distal end 612, for example. The tear strip 632 may be grasped by the user and pulled to split the tubular member 615 and thus allow the outflow blocking sleeve 600 to be removed laterally from the blood pump 50 and/or elongate shaft 12 of the catheter 10 by passing the blood pump 50 and/or elongate shaft 12 of the catheter 10 through the gap formed from the removal of the tear strip 632. In some instances, the tear strip 632 may include a tab 634, configured to be grasped by the user. In some instances, the tab 634 may extend proximally of the proximal end 614 and/or distally from the distal end 612, if desired.

Another illustrative embodiment of the outflow blocking sleeve 200, depicted as outflow blocking sleeve 700, is shown in FIG. 14. The outflow blocking sleeve 700 may be similar to the outflow blocking sleeve 200 in many aspects. For example, the outflow blocking sleeve 700 may be a tubular member 715 having a distal end 712, a proximal end 714, and an inner wall surface 718 defining a lumen extending therethrough. The outflow blocking sleeve 700 may include a tapered region 716 located between a distal end region extending to the distal end 712 and a reduced diameter proximal end region extending to the proximal end 714. The outflow blocking sleeve 700 may include a gripping structure on an exterior surface of tubular member 715 to facilitate gripping and thereby manipulating the outflow blocking sleeve 700 during use. For example, the gripping structure may include one or more, or a plurality of raised ridges 740, as shown. In some instances, the raised ridge(s) 740 may extend circumferentially and/or helically around the tubular member 715. In other instances, the raised ridge(s) 740 may be one or more bumps or protuberances raised above the remainder of the outer surface of the tubular member 715. In other instances, the gripping structure may be one or more, or a plurality of grooves, recesses, tabs, flaps, or other features to enhance gripping or otherwise manipulating the outflow blocking sleeve 700.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A system, comprising:
an introducer sheath including a hub, an elongate shaft extending distally from the hub, and an elastomeric seal positioned in the hub;
a catheter including an elongate shaft and a percutaneous blood pump positioned at a distal end of the elongate shaft, the percutaneous blood pump including a blood inlet, a blood outlet, and an impeller assembly positioned therebetween to pump blood from the blood inlet to the blood outlet during use; and
an outflow blocking sleeve removably surrounding the blood outlet;
wherein the blood pump is sized to be passed distally through the elastomeric seal into a lumen of the elongate shaft of the introducer sheath while the outflow blocking sleeve is prevented from passing through the elastomeric seal during advancement of the blood pump through the elastomeric seal.

2. The system of claim 1, wherein the outflow blocking sleeve includes a distal end region having an inner diameter and a proximal end region having a reduced inner diameter less than the inner diameter of the distal end region.

3. The system of claim 2, wherein the distal end region tightly surrounds the blood outlet to occlude blood flow therefrom and the proximal end region extends over a distal portion of the elongate shaft of the catheter.

4. The system of claim 1, wherein the outflow blocking sleeve has a length of 7 cm or less.

5. The system of claim 1, further comprising a guidewire extending out of an outflow window of the blood outlet and extending proximally therefrom along an exterior of the blood pump.

6. The system of claim 5, wherein the outflow blocking sleeve surrounds the guidewire extending along the exterior of the blood pump.

7. The system of claim 1, wherein the outflow blocking sleeve includes a preferential separation line extending along a length of the outflow blocking sleeve.

8. The system of claim 1, wherein the outflow blocking sleeve includes a strip of material configured to separate from a tubular wall of the outflow blocking sleeve when pulled.

9. The system of claim 1, wherein the outflow blocking sleeve includes a flared distal end configured to abut a proximal face of the elastomeric sleeve.

10. The system of claim 1, wherein the outflow blocking sleeve includes a bulbous distal end configured to abut a proximal face of the elastomeric sleeve.

11. A system, comprising:
a catheter including an elongate shaft and a percutaneous blood pump positioned at a distal end of the elongate shaft, the percutaneous blood pump including a blood inlet, a blood outlet, an impeller positioned therebetween to pump blood from the blood inlet to the blood outlet during use, and a motor housing positioned proximal of the impeller, the motor housing including a motor to rotationally drive the impeller; and
an outflow blocking sleeve tightly surrounding the blood outlet and extending proximally therefrom to surround the motor housing;
wherein the outflow blocking sleeve is configured to prevent blood egress from the blood outlet during advancement of the blood pump into a vasculature at a vascular access site.

12. The system of claim 11, wherein the outflow blocking sleeve includes a distal end region having an inner diameter and a proximal end region having a reduced inner diameter less than the inner diameter of the distal end region.

13. The system of claim 12, wherein the distal end region tightly surrounds the blood outlet to occlude blood flow therefrom and the proximal end region extends over a distal portion of the elongate shaft of the catheter proximal of the motor housing.

14. The system of claim 11, wherein the outflow blocking sleeve has a length of 7 cm or less.

15. The system of claim 11, further comprising a guidewire extending out of an outflow window of the blood outlet and extending proximally therefrom along an exterior of the motor housing, wherein the outflow blocking sleeve surrounds the guidewire extending along the exterior of the motor housing.

16. A method of percutaneously inserting a blood pump into a vasculature at a vascular access site, the method including:
advancing a catheter, including the blood pump positioned at a distal end of an elongate shaft of the catheter, into an introducer sheath having an elastomeric seal, the blood pump including a blood inlet, a blood outlet, and an impeller assembly positioned therebetween to pump blood from the blood inlet to the blood outlet during use,
advancing the blood pump through the elastomeric seal while an outflow blocking sleeve occludes the blood outlet when the blood outlet is located proximal of the elastomeric seal and the blood inlet is located distal of the elastomeric seal such that blood egress from the blood outlet is prevented; and
further advancing the blood pump through the elastomeric seal while the outflow blocking sleeve abuts a proximal face of the elastomeric sleeve and remains exterior of the elastomeric seal such that the blood outlet passes through the elastomeric seal to a location distal of the elastomeric seal.

17. The method of claim 16, wherein the blood pump is advanced over a guidewire extending out of an outflow window of the blood outlet and the guidewire extends proximally therefrom along an exterior of the blood pump.

18. The method of claim 17, wherein the outflow blocking sleeve surrounds the guidewire extending along the exterior of the blood pump.

19. The method of claim 16, further comprising laterally removing the outflow blocking sleeve from the catheter after the blood outlet of the blood pump is advanced to a location distal of the elastomeric seal.

20. The method of claim 19, wherein the outflow blocking sleeve is splittable along its length to separate the outflow blocking sleeve from the catheter.
